(19) United States
(12) Patent Application Publication
(10) Pub. No.: US 2002/0055686 A1
(43) Pub. Date: May 9, 2002

(54) Method of evaluation and selection of absorbent article

(76) Inventors: Takayuki Hisanaka, Kagawa-ken (JP); Yuri Imamura, Kagawa-ken (JP)

Correspondence Address: PAULA CAMPBELL EVANS, PALMER & DODGE LLP, One Beacon Street, Boston, MA, 02108, US

(21) Appl. No.: 09/887,962

(22) Filed: Jun. 22, 2001

(30) Foreign Application Priority Data
Sep. 1, 2000 (JP) 265697/2000

(51) Int. Cl. A61B 5/04

(52) U.S. Cl. 600/544

(57) ABSTRACT

The present invention relates to a method of evaluating and selecting an absorbent article comprising using the brain wave fluctuations of the wearer as a measure. The method enables evaluating and selecting absorbent articles such as disposable diapers and sanitary napkins conforming to the user's physiological and psychological requirements.

### DESCRIPTION

1. Technical Field

The present invention relates to a method of evaluating and selecting absorbent articles such as disposable diapers and sanitary napkins. More particularly, the present invention relates to a method of evaluating and selecting an absorbent article most appropriate for physiological and psychological requirements of users by measuring a brain wave and using the fluctuation of the brain wave of the user as a measure.

2. Background Art

At present, absorbent articles such as disposable diapers, sanitary napkins, vaginal discharge liners, and breast milk pads are classified for sale in the market according to the dimensions (length, thickness, etc.), configuration, absorption amount, price, and the like.

For this reason, users select and purchase these absorbent articles according to the dimensions, configuration, absorption amount, price, and the like.

In addition, in the absence of expert sales personnel in shops, the purchasers have to select and purchase the articles displayed on the shelves according to the dimensions, configuration, absorption amount, and the like described on the packages.

For example, when buying a diaper for infants, the purchaser must select the articles according to the size or the body weight (e.g. the size for new-born babies, S, M, L, etc.) and the type classified according to the infant's ability to stand and walk or not (e.g. a type of assembling using tapes, or shorts type). When purchasing a diaper for adults, the waist size (S, M, L, etc.) and the form maybe the measure for selection. Sanitary napkins are selected according to the amount of absorption and length (depending on the amount of vaginal discharge) and the form (with or without flaps).

Because the users usually purchase sanitary napkins themselves, they can select the articles most suited for their condition and favorite (tactile sense, form, absorption amount, etc.) through their experience. In addition, the sanitary napkin users can cope with troubles caused by the goods by themselves by receiving therapeutic measures in hospital, etc.

However, a purchaser is not usually the user in the case of diapers for infants or adults. Therefore, the goods are generally selected through the judgment of the purchaser, who is a care person or a helper, according to the size, leak-proof properties, etc. This tendency is particularly strong in the case of infants not possessing sufficient language and exercise capability or elderly persons with dementia symptoms.

Said selection is thus not necessarily in accord with the physiological or psychological requirements of the users. Selected goods may impart an unacceptable feeling of use (tactile sense, tightness, movability) and may result in externally induced diseases such as diaper rash (contact dermatitis) or may cause a psychic stress in the users. If such an absorbent article is continuously used, immune functions of the user may deteriorate due to a latent psychic stress.

Under such commercial circumstances, efforts of personnel involved in the development and manufacturing of absorbent articles may be directed to the purchaser's taste and convenience, and the development of product performance may not be in line with the user's requirements.

However, no method for correcting such a situation has conventionally been developed. There have been no appropriate measures used in the evaluation and selection of absorbent articles conforming to the user's comfort and tactile sensation.

### DISCLOSURE OF THE INVENTION

An object of the present invention is therefore to solve the above problems in the conventional technology. A specific object of the present invention is to provide a method of evaluating and selecting absorbent articles such as disposable diapers and sanitary napkins conforming to the user's physiological and psychological requirements.

More specifically, an object of the present invention is to provide a method enabling developers and manufacturers of diapers to evaluate and select diapers closer to the user's physiological and psychological requirements such as wearing comfort and a method enabling purchasers to evaluate and select diapers more appropriate to the user's physiological and psychological requirements.

As a result of extensive studies with an objective of solving the above problems, the present inventors have found that the absorbent articles can be evaluated and selected by using fluctuation of the brain wave as a measure. This finding has led to the completion of the present invention.

In the investigation of various measures which can objectively estimate physiological and psychological conditions of the diaper users, the present inventors paid attention to brain waves and found that the degree of comfort of a diaper is related to fluctuations in the brain waves of the wearer. Specifically, the inventors found that the type of brain wave fluctuations of the wearer of an absorbent article when the wearer feels comfortable differs from the type of brain wave fluctuations when he or she feels uncomfortable.

Therefore, the present invention relates to:

(1) A method of evaluating suitability of absorbent articles comprising using the brain wave fluctuation of the wearer as a measure,

(2) A method of selecting an absorbent article suitable for a person wearing the article comprising using the brain wave fluctuations of the wearer as a measure,

(3) The method described in (1) or (2) above, wherein the brain wave fluctuations are α-wave fluctuations, and

(4) The method described in any one of (1) to (3) above, comprising measuring the power spectrum of brain waves of a person wearing an absorbent article before and after urination to determine the fluctuation in the power spectrum depending on time and using said fluctuation as the measure.

The present invention exhibits the special effect that an absorbent article satisfying physiological and psychological requirements of the wearer such as degree of comfort can be objectively and easily selected and evaluated by using the brain wave fluctuations of the wearer as a measure.

### DETAILED DESCRIPTION OF THE INVENTION AND PREFERRED EMBODIMENT

The present invention will be explained in more detail in the following description, which is not intended to be limiting of the present invention.

The absorbent articles in the present invention include disposable diapers, sanitary napkins, vaginal discharge liners, and breast milk pads. In addition, the present invention can also be applied to underwear shorts for incontinence and the like.

Brain waves can be used as a measure of the central nerve system and are classified according to frequency into a δ-wave with a frequency of less than 4 Hz, a θ-wave with a frequency range of not less than 4 Hz to less than 8 Hz, an α-wave with a frequency range of not less than 8 Hz to less than 13 Hz, and a β-wave with a frequency range of not less than 13 Hz to 30 Hz, and the like. A δ-Wave is not seen in a normal adult when he or she is awake, but generates during deep sleep according to a decrease in the brain function. A θ-Wave generates during REM sleep. An α-Wave generates particularly in the back or top of the head when a person is not strained or his mental activity is not active, for example when a person shuts the eyes or is at rest, and a β-wave generates from the entire cortex when a person is awake.

In the present invention, the inventors paid particular attention to an α-wave which is associated with a person's relaxed state and investigated the relationship between the α-wave and the degree of comfort of absorbent articles such as a diaper.

Brain waves can be measured by using a conventional brain wave measuring apparatus such as a polygraph or an electroencephalograph. An example of the measuring method will be described below (see also “The Handbook of Human Factors and Ergonomics”, edited by The Japan Society of Physiological Anthropology, Instrumentation Research Department, Gihodoshuppan Co., Ltd., pages 113-134).

[Method of Measuring Brain Waves]

A plate electrode made of silver-silver chloride is used as the leading electrode for brain waves.

The surface of the head skin to which the electrode is attached is cleaned with cotton containing alcohol, and the electrode to which electrode paste has been applied is attached according to the International Electrode Arrangement Method (10-20 method) . An electric potential of each electrode on the surface of the skin is amplified by an AC amplifier. The output signals are sent to an A/D converter and converted to digital signals. The data obtained is stored in media such as DAT, MO, etc., and the resulting power spectra are analyzed.

[Test of Wearing Absorbent Article]

The power spectrum analysis was carried out at 13 points on the head skin to investigate the relationship between the brain wave and the sensation of the absorbent article wearer.

The test method and the results are shown in Table 1.

(Absorbent Article)

The following two pants-type diapers are used as the absorbent articles.

TABLE 1

| | Rate of | Rewet | Absorbed | Retained |
|---|---|---|---|---|
| | absorption | amount | amount | water amount |
| | (sec) | (g) | (g/p) | (g/p) |
| Diaper I | 30.0 | 6.4 | 807.0 | 384.0 |
| Diaper II | 27.2 | 76.7 | 504.0 | 177.0 |

Note:

Rate of absorption: the time required for the total amount of 200 ml of a physiological saline solution added to the diaper is absorbed through the top sheet.

Rewet amount: the amount of a physiological saline solution absorbed in a filter paper placed on the top sheet of a diaper under a load of 35 g/cm² five minute after 200 ml of a physiological saline solution is added.

Absorbed amount: the value determined by subtracting the weight of a diaper before absorbing water from the weight of the diaper that was dipped in a physiological saline solution for 30 minutes, removed from the solution, and allowed to stand under a load of 35 g/cm² for 20 minutes to let water to escape. (g/p indicates the amount of water absorbed per one diaper.)

The amount of retained water: the value determined by subtracting the weight of a diaper before absorbing water from the weight of the diaper after absorbing water according to the above procedure of determining the amount of water absorption and removing the water by centrifugation at 150 G for 90 seconds.

The diaper I is a sample representing a diaper exhibiting good performance in the rate of absorption, rewetting amount, amount of water absorption, and amount of retained water, whereas diaper II is a sample exhibiting poor performance in all of these tested items.

(Wearing Test Method)

Seven adult panelists performed the wearing test. The panelists wore the diaper I or II in a room at 28° C. and 60% RH. After 25 minutes, simulated urine at 37° C. was applied between the diaper and the thigh using a silicon tube. The panelists continued to wear the diaper for 25 minutes.

(Results)

The α-wave at 13 points on the head skin of each panelist was measured according to the above method of brain wave measurement.

The power spectrum of each point was verified by t-test regarding to (1) after wearing the diaper and immediately after urination, and (2) at 6 minutes after urination and at 19 minutes after urination. The number of points among 13 points, in which the verified values obtained (1) after wearing the diaper and immediately after urination and (2) at 6 minutes after urination and 19 minutes after urination differed significantly (level of verified difference is less than 5%) is shown in Table 2.

TABLE 2

| | (1) The number of points | |
|---|---|---|
| | in which the power | (2) The number of points |
| | spectrum value after | in which the power |
| | wearing the diaper | spectrum value 6 minutes |
| | differed significantly | after urination differed |
| | from the power spectrum | significantly from the |
| | value immediately after | power spectrum value 19 |
| | urination | minutes after urination |
| Diaper I | 6 | 13 |
| Diaper II | 7 | 3 |

As can be seen in Table 2, there was no difference between the number of points in which the α-wave power spectrum after wearing the diaper and immediately after urination significantly differed in the diaper I with good performance and the diaper II with poor performance, whereas the number of points in which the α-wave power spectrum at 6 minutes after urination and 19 minutes after urination differed significantly was remarkably greater in the diaper I than in the diaper II.

Activity of the central nerve system can be said to be free at the points at which the α-wave exhibited a significant difference over time after urination. Because the wearers of diaper I did not have a badly wetted sensation after urination, their brain maintained free activity and the α-waves fluctuated more freely without any restriction by the brain, resulting in a larger number of points at which the brain wave fluctuation exhibited significant difference. On the other hand, the central nerve system could not maintain free activity at the points in which the α-wave exhibited no significant difference over time after urination, because the central nerve system at these points continuously stimulated by an impaired wet sensation. Because the α-waves of the brain of wearers of diaper II were restrained due to the failure to maintain a free state of activity, the α-waves could not freely fluctuate, resulting in a small number of points at which the brain wave fluctuation exhibited a significant difference.

The above results indicate that a diaper causing fluctuation in the brain wave due to the change in the wearing environment can be said to be a more comfortable diaper. In addition, a diaper causing fluctuation in the brain waves can be said to be a diaper capable of recovering comfortable condition after urination within a short period of time.

As a result, a diaper with good performance is judged to exhibit high brain wave fluctuations. Therefore, it is possible to evaluate and select a diaper suitable for a specific wearer by measuring his or her brain wave fluctuations after urination.

These results indicate that the degree of brain wave fluctuations is not only applicable to evaluation of diapers in the development and manufacture, but also usable as a measure for selecting a diaper imparting a comfortable feeling to users such as infants and aged persons who cannot express their own feelings.

If these results are applied, it is possible to provide diapers more appropriate to the users (wearers) by developing and manufacturing diapers exhibiting high brain wave fluctuations.

Although the present invention has been described referring to the relationship between the absorption performance of a diaper and the brain wave fluctuations in the above description, an absorbent article most appropriate for the user in terms of other properties such as tactile sense, tightness, and movability can also be selected by determining the brain wave fluctuations in the same manner.

In addition, although the relationship between the absorbent article and the brain wave fluctuations is determined using a diaper as an example of the absorbent article in the above description, the same results can also be expected for other absorbent articles.

### EXAMPLES

The present invention will now be described by way of examples, which should not be construed as limiting the present invention.

Three types of diapers I-III with different absorption performance (see Table 3) were used by seven panelists.

The panelists wore the diapers in a room at 28° C. and 60% RH. After 25 minutes, simulated urine at 37° C. was discharged between the diaper and the thigh through a silicon tube. The panelists continued to wear the diaper for 25 minutes.

The frequency of brain waves at 13 points on each head of seven panelists was analyzed. The power spectrum of each point was verified by t-test regarding to (1) after wearing the diaper, immediately after urination, and (2) at 6 minutes after urination and at 19 minutes after urination. The number of points among 13 points, in which the verified values obtained (1) after wearing the diaper and immediately after urination and (2) at 6 minutes after urination and 19 minutes after urination differed significantly (level of verified difference is less than 5%) is shown in Table 4.

[Degree of Comfort]

Degree of comfort felt by the wearers after urination was evaluated according to the following criteria of three levels.

3: Almost no change in the wearing sensation after urination as compared with before urination.

2: The wearing sensation was slightly more uncomfortable after urination as compared with before urination.

1: The wearing sensation was uncomfortable after urination as compared with before urination.

The values averaged for the seven panelists are shown in Table 5.

[Observation of Skin Conditions]

Aged persons requiring care wore the diapers, which were replaced seven times a day on average. After five days, the skin conditions were observed by the naked eye. The results are also shown in Table 5.

TABLE 3

| | Rate of | Rewet | Absorbed | Retained |
|---|---|---|---|---|
| | absorption | amount | amount | water amount |
| | (sec) | (g) | (g/p) | (g/p) |
| Diaper I | 30.0 | 6.4 | 807.0 | 384.0 |
| Diaper II | 27.2 | 76.7 | 504.0 | 177.0 |
| Diaper III | 28.6 | 43.6 | 638.0 | 256.0 |

TABLE 4

| | (1) The number of points | (2) The number of points |
|---|---|---|
| | in which the power | in which the power |
| | spectrum value after | spectrum value 6 |
| | wearing the diaper | minutes after urination |
| | differed significantly | differed significantly |
| | from the power spectrum | from the power spectrum |
| | value immediately after | value 19 minutes after |
| | urination | urination |
| Diaper I | 6 | 13 |
| Diaper II | 7 | 3 |
| Diaper III | 6 | 5 |

TABLE 5

| | Degree of comfort | Condition of the skin |
|---|---|---|
| Diaper I | 2.7 | No abnormalities |
| Diaper II | 1.1 | Erythema produced |
| Diaper III | 1.9 | Slight erythema produced |

Based on the results of Tables 4 and 5, the number of points among the 13 points on the wearer's head in which the α-wave exhibited a significant fluctuation after urination has been confirmed to be related to degree of comfort of the wearers of the diaper. Specifically, a diaper with a larger number of points in which the brain waves significantly fluctuated due to the change in the diaper wearing environment was confirmed to impart greater comfort to the wearer and to maintain a good skin condition.

### INDUSTRIAL USEFULNESS

The use of brain wave fluctuations as a measure of evaluation according to the present invention ensures objective evaluation of an absorbent article possessing appropriate functions. The present invention therefore can be applied to development of an absorbent article possessing appropriate functions. In addition, an absorbent article satisfying the wearer's desired degree of comfort can be selected and purchased by determining the wearer's brain wave fluctuations.

1. A method of evaluating suitability of absorbent articles comprising using the brain wave fluctuations of the wearer as a measure.

2. A method of selecting an absorbent article suitable for a person wearing the article comprising using the brain wave fluctuations of the wearer as a measure.

3. The method according to claim 1 or 2, wherein the brain wave fluctuations are α-wave fluctuations.

4. The method according to any one of claims 1 to 3, comprising measuring the power spectrum of brain wave of a person wearing an absorbent article before and after urination to determine the fluctuation in the power spectrum depending on time and using said fluctuation as the measure.
